(19) United States
(12) Patent Application Publication
(10) Pub. No.: US 2020/0246589 A1
(43) Pub. Date: Aug. 6, 2020

(54) Device and Method for Starting a Catheter in a Female Patient

(71) Applicant: Nancy Starr, Little Torch Key, FL (US)

(72) Inventor: Nancy Starr, Little Torch Key, FL (US)

(21) Appl. No.: 16/782,159

(22) Filed: Feb. 5, 2020

Related U.S. Application Data
(60) Provisional application No. 62/801,171, filed on Feb. 5, 2019.

(51) Int. Cl. A61M 25/01

(52) U.S. Cl. CPC A61M 25/0111; A61M 2210/1092; A61M 2210/1475; A61M 2210/1483

(57) ABSTRACT

A device and method for preparing a female patient to receive a catheter includes a female catheter starter cone that is shaped and constructed for insertion into the urethral fold of a women's body that holds the labia majora and labia minora out of the way so that a nurse can visually identify the urethral opening and to facilitate single handed insertion of the catheter tube.

### REFERENCE TO RELATED APPLICATIONS

This application claims the priority of provisional patent application U.S. Ser. No. 62/801,171 filed Feb. 5, 2019 titled Female Catheter Starting Cone and which is incorporated herein by reference in its entirety.

### BACKGROUND OF THE INVENTION

This invention relates generally to nursing devices and, more particularly, to a starter cone that flexes for insertion into a woman's urethral fold in the urogenital groove so as to expose the woman's urethra while a catheter is inserted therein.

Almost everything a nurse does is governed by a written step-by-step procedure for good reasons, including so that no step gets omitted, to formalize nurse training, and so that good practices become routine. Insertion of a catheter is a frequent nursing task, such as before a patient goes into surgery or has a urinary retention for any reason. This may occur several times per day in many nursing roles.

However, starting a catheter in a female patient is not an easy or trivial procedure for nurses—even for experienced nurses. The exact position and appearance of the urethral opening is not always the same and some women, particularly, women who are obese, may have a urethral opening that is covered by the multiple folds of a woman's urogenital area. Similarly, patients who are disabled or are incapable of cooperating for proper positioning are a particular challenge to the procedure. This results in a nurse sometimes having to use both hands to effectively clean this area, locate the urethral opening, and then to insert the tube of a catheter into the urethral opening.

Therefore, it would be desirable to have a device and method that includes a female catheter starter cone uniquely shaped and constructed for insertion into the urethral fold area of a women's body that holds the labia majora and labia minora out of the way so that a nurse can visually identify the urethral opening and to facilitate single handed insertion of the catheter tube.

### SUMMARY OF THE INVENTION

A device and method for preparing a woman to be catheterized according to the present invention includes a catheter starter cone that includes a side wall having a continuous and frusto-conical configuration that defines a first open end and a second open end opposite the first open end. Preferably, the second open end has a diameter that is larger than a diameter of the first open end. The continuous side wall is constructed of a resilient material that is movable between (1) a pinched configuration in which a first side wall of the continuous side wall is proximate a second side wall of the continuous side wall for positioning the open end of the continuous side wall into a urogenital area of the female patient and (2) a released configuration in which the first side wall is displaced from the second side wall such that the open end of the continuous side wall holds the labia majora and labia minora of the female patient's urogenital area out of the way so that the urethral opening and vagina of the female patient are visible.

Therefore, a general object of this invention is to provide a device and method for preparing a female patient to be catheterized.

Another object of this invention is to provide a device and method that includes a female catheter starter cone that holds the labia majora and labia minora out of the way so that the urethral opening can be visually identified.

Another object of this invention is to provide the female catheter starter cone, as aforesaid, that pushes the multiple folds of a women's urogenic area to the side so that the urethral opening and vagina are seen more clearly.

Still another object of this invention is to provide the female catheter starter cone, as aforesaid, that is flexible such it may be pinched to a compressed configuration for insertion and which is resilient to push the urogenic tissues to the side when uncompressed.

Other objects and advantages of the present invention will become apparent from the following description taken in connection with the accompanying drawings, wherein is set forth by way of illustration and example, embodiments of this invention.

### BRIEF DESCRIPTION OF THE DRAWINGS

FIG. 1 is a top perspective view of a female catheter starter cone according to a preferred embodiment of the present invention;

FIG. 2 is side perspective view of the female catheter starter cone as in FIG. 1;

FIG. 3 is a top view of the female catheter starter cone as in FIG. 1;

FIG. 4 is a side view of the female catheter starter cone as in FIG. 1;

FIG. 5 is an end view of the female catheter starter cone as in FIG. 1;

FIG. 6 is a bottom view of the female catheter starter cone as in FIG. 1;

FIG. 7 is a sectional view taken along line 7-7 of FIG. 6; and

FIG. 8 is an exploded view of a female catheter starter cone according to the present invention.

### DESCRIPTION OF THE PREFERRED EMBODIMENT

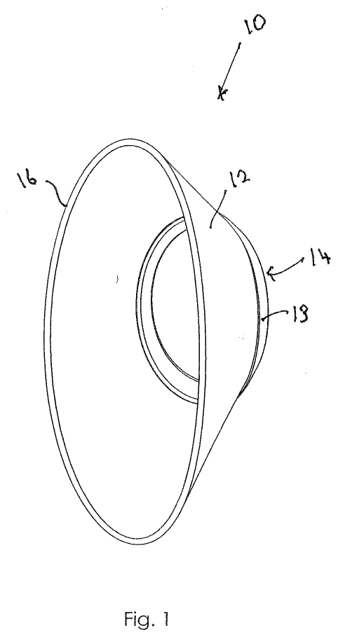

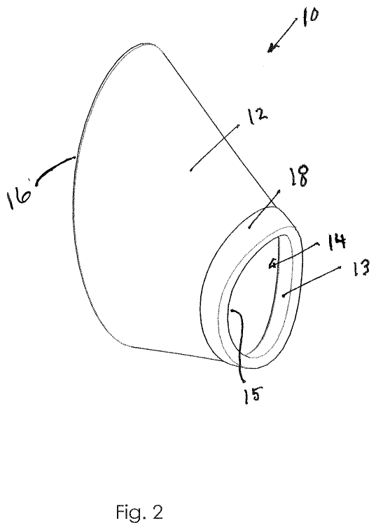

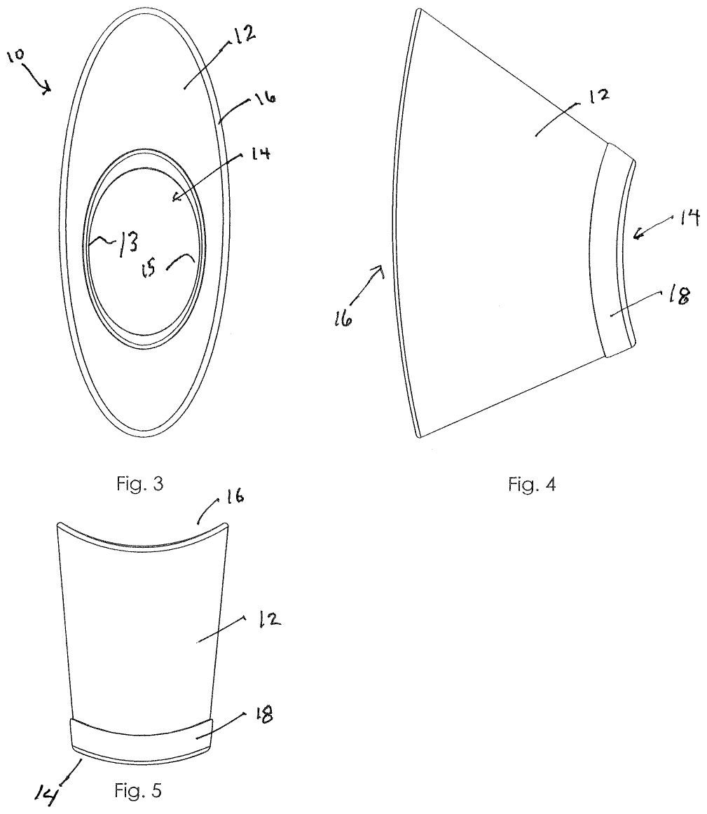

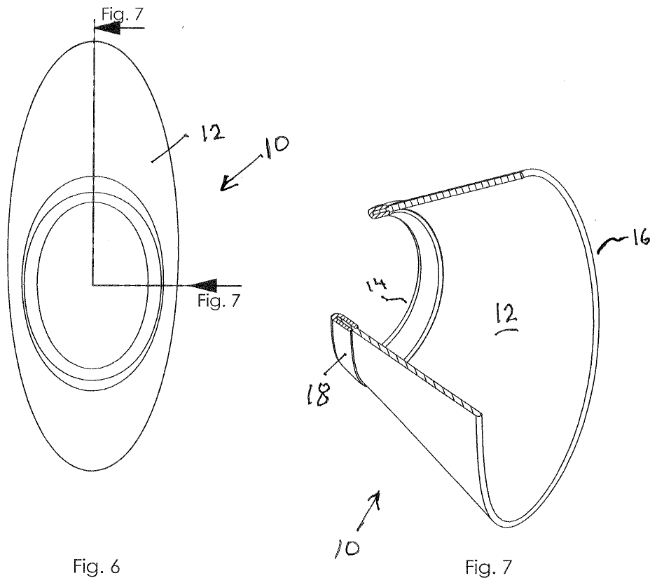

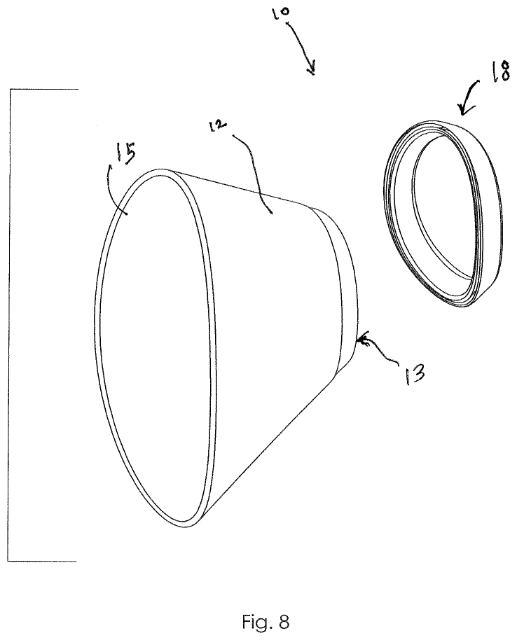

A female catheter starter cone according to the present invention will be described with reference to FIGS. 1 to 8 of the accompanying drawings.

The starter cone 10 has a continuous side wall 12 forming a first open end 13 and an opposed open second end 15, the continuous side wall 12a having a frusto-conical configuration. The starter cone 10 may be constructed of a resilient material. The concept of resilience is known in material science or physics as the tendency or capability to bounce back or return to an initial configuration after having been deformed. Scientifically, resilience is a quantifiable ability of a material to absorb energy (or to become loaded with energy) and then to shed that energy when the load is removed (i.e. is unloaded). In the present instance, the continuous side wall 12 may be constructed of a resilient material, such as rubber, a series of rubber threads woven together (i.e. elastic material), spandex, spring steel, similar polymer, or the like. More particularly, a first open end 13 has a frusto-conical opening defining a first dimension. The second open end 15 also has a frusto-conical opening defining a second dimension. The first and second openings may have configurations that are congruent with one another (i.e. same shape but different sizes) and, in some embodiments, may be concentric. The second dimension may be 1½ to 2 times larger than the first dimension. In an embodiment, the first end may have a dimension that is about 40 mm by 20 mm (i.e. length by width) and the second end may have a dimension that is about 100 mm by 40 mm. Other dimensions may also be possible.

The intention is that, when initially pinched or squeezed together, the first open end 13 of the starter cone 10 may be inserted or positioned to become lodged in the genital fold area of a woman's body (also referred to as a female patient) and then to push outwardly on the folds thereof when the starter cone 10 is released and allowed to move resiliently to a non-compressed configuration. This area may also be referred to as a woman's urogenital area. Stated another way, the continuous side wall 12 is movable between (1) a pinched configuration in which a first side wall 14 is proximate (i.e. near) a second side wall 16 so that the first open end 13 may be inserted or otherwise positioned to the inside of the folds of the female patient's urogenital area and (2) a released configuration in which the first side wall 14 is displaced from the second side wall 16 such that the continuous side wall 12 pushes and holds the labia majora and labia minora of the female patient's urogenital area out of the way (of vision). Then, the nurse is able to look through the second open end 15 to visually identify the urethral opening of the female patient and target insertion of a catheter (not shown). By frictional forces alone, the starter cone 10 will stay in place so that the nurse's hands are free to insert a free end of the catheter.

In addition, the starter cone 10 may include a foam ring 18 coupled to and positioned exteriorly about the first open end 13 of the side wall 12. The foam ring 18 may provide more comfort and secure positioning of the first open end 13 when the starter cone 10 is positioned in the genital area of the patient. More particularly, the foam ring 18 may provide a material or coating that is more comfortable when bearing against the genital tissues of the patient as described above. It is understood that the foam ring 18 is constructed of a resilient material similar to that of the continuous side wall 12 and also a material likely to grip the urogenital tissues and not slip.

It is understood that while certain forms of this invention have been illustrated and described, it is not limited thereto except insofar as such limitations are included in the following claims and allowable functional equivalents thereof.

1. A catheter preparatory device for treating a female patient in need of catheterization, comprising:
a catheter starter cone that includes a side wall having a continuous and frusto-conical configuration that defines a first open end and a second open end opposite said first open end;
wherein said second open end has a diameter that is larger than a diameter of said first open end; and
wherein said continuous side wall is constructed of a resilient material that is movable between (1) a pinched configuration in which a first side wall of the continuous side wall is proximate a second side wall of the continuous side wall for positioning said open end of said continuous side wall into a urogenital area of the female patient and (2) a released configuration in which said first side wall is displaced from said second side wall such that said open end of said continuous side wall holds the labia majora and labia minora of the female patient's urogenital area out of the way so that the urethral opening and vagina of the female patient are visible.

2. The catheter preparatory device as in claim 1, wherein:
said first side wall is positioned close to said second side wall at said pinched configuration; and
said first side wall is extended away from said second side wall at said released configuration.

3. The catheter preparatory device as in claim 1, wherein:
said first side wall is parallel to said second side wall at said pinched configuration; and
said first side wall and said second side wall form a generally circular configuration is at said released configuration.

4. The catheter preparatory device as in claim 1; wherein a diameter of said second open end is about 1 to 2½ times larger than a diameter of said first open end.

5. The catheter preparatory device as in claim 1, wherein:
said first open end has a dimension that is about 40 mm long and about 20 mm wide; and
said second open end has a dimension that is about 100 mm long and about 40 mm wide.

6. The catheter preparatory device as in claim 1, wherein said resilient material is one of rubber, a plurality of rubber stands woven together, spandex, or spring steel.

7. The catheter preparatory device as in claim 1, further comprising foam ring coupled to an exterior surface of said first open end, said foam ring being constructed of a resilient material having a skin gripping surface.

8. A method of using a catheter preparatory device to treat a female patient in need of catheterization, said method, comprising:
providing a catheter starter cone that includes a continuous side wall having a frusto-conical configuration constructed of a resilient material and that defines a first open end and a second open end opposite said first open end;
wherein said second open end has a diameter that is larger than a diameter of said first open end; and
moving said continuous side wall between (1) a pinched configuration in which a first side wall of the continuous side wall is proximate a second side wall of the continuous side wall for positioning said open end of said continuous side wall into a urogenital area of the female patient and (2) a released configuration in which said first side wall is displaced from said second side wall such that said open end of said continuous side wall holds the labia majora and labia minora of the female patient's urogenital area out of the way so that the urethral opening and vagina of the female patient are visible.

9. The method as in claim 8, wherein:
said first side wall is positioned close to said second side wall at said pinched configuration; and
said first side wall is extended away from said second side wall at said released configuration.

10. The method as in claim 8, wherein:
said first side wall is parallel to said second side wall at said pinched configuration; and
said first side wall and said second side wall form a generally circular configuration is at said released configuration.

11. The method as in claim 8; wherein a diameter of said second open end is about 1 to 2½ times larger than a diameter of said first open end.

12. The method as in claim 8, wherein:
said first open end has a dimension that is about 40 mm long and about 20 mm wide; and
said second open end has a dimension that is about 100 mm long and about 40 mm wide.

13. The method as in claim 8, wherein said resilient material is one of rubber, a plurality of rubber stands woven together, spandex, or spring steel.

14. The method as in claim 8, further comprising coupling a foam ring to an exterior surface of said first open end, said foam ring being constructed of a resilient material having a skin gripping surface.
